(19) United States
(12) Patent Application Publication
(10) Pub. No.: US 2022/0208309 A1
(43) Pub. Date: Jun. 30, 2022

(54) PREDICTION OF SIDE-CHAIN DEGRADATION IN POLYMERS THROUGH PHYSICS BASED SIMULATIONS

(71) Applicant: Genentech, Inc., South San Francisco, CA (US)

(72) Inventors: Saeed Izadi, South San Francisco, CA (US); Flaviyan Jerome Irudayanathan, South San Francisco, CA (US)

(73) Assignee: Genentech, Inc., South San Francisco, CA (US)

(21) Appl. No.: 17/587,760

(22) Filed: Jan. 28, 2022

Related U.S. Application Data
(63) Continuation of application No. PCT/US2020/044222, filed on Jul. 30, 2020.
(60) Provisional application No. 62/882,155, filed on Aug. 2, 2019.
(60) Provisional application No. 62/979,507, filed on Feb. 21, 2020.

(51) Int. Cl. G16C 10/00; G16C 20/10; G16B 15/30

(52) U.S. Cl. CPC G16C 10/00; G16B 15/30; G16C 20/10

(57) ABSTRACT

The present disclosure relates to polypeptide therapeutics, and in particular to techniques for predicting side-chain degradation in polymers through physics based simulations. Particularly, aspects of the present disclosure are directed to generating a representation of a polymer having one or more side chains, performing a molecular-dynamics simulation using the representation to obtain a set of polymer conformations, determining, for each polymer conformation, one or more spatial characteristics of the polymer while in the polymer conformation, identifying, based on the one or more spatial characteristics, an incomplete subset of the set of polymer conformations estimated to undergo one or more reactions of a particular type, and estimating, based on a size of the incomplete subset, a probability of a reaction in which the polymer is a reactant and a particular other molecule is a product.

### CROSS-REFERENCE TO RELATED APPLICATIONS

The present application is a continuation of International Patent Application No.: PCT/US2020/044222, dated Jul. 30, 2020, which claims priority and benefit from U.S. Provisional Application No. 62/882,155, filed on Aug. 2, 2019 and U.S. Provisional Application No. 62/979,507, filed on Feb. 21, 2020, the entire contents of which are incorporated herein by reference for all purposes.

### FIELD

The present disclosure relates to polypeptide therapeutics, and in particular to techniques for predicting side-chain degradation in polymers through physics based simulations.

### BACKGROUND

Multiple types of reactions may result in a chemical degradation of a molecule. For example, chemical degradation may occur as a result of isomerization or deamidation. In the context of biologics, chemical degradation may reduce availability of a therapeutic and/or reduce likelihood of triggering a target biological effect. For example, aspartate isomerization can result in a loss of potency, and isoaspartate formation has been linked to Alzheimer's disease. It would be advantageous to be able to detect the likelihood of chemical degradation for a given molecule early during the research and development process. By detecting chemical degradation (for example) the development of a molecule that is likely to degrade may be avoided or coupled with an approach to mitigate the undesired effects of the degradation. One approach for predicting whether a given molecule will degrade is to execute a simulation. However, chemical degradation can include sub-atomic interactions, covalent-bond formation and covalent-bond breakage, and conventional molecular-dynamic simulations are not configured to model these types of events.

### SUMMARY

In some instances, techniques are provided that predict the likelihood that a given polymer molecule (e.g., a polypeptide molecule) will degrade to a particular potential degraded product. A given polymer molecule may have any of multiple conformations. Thus, an experiment (e.g., a computational experiment, such as one using a simulation or artificial intelligence) may be conducted that predicts a likelihood that the polymer will transition into a conformation likely to undergo reactions to produce the particular potential degraded product. This prediction can include identifying spatial features that make a polymer susceptible to particular reactions and using a molecular dynamics simulation to predict a likelihood that the polymer will transition to a conformation that has those spatial features.

In some instances, a computer-implemented method is provided. A representation of a polymer having one or more amino acids can be generated. A molecular-dynamics simulation can be performed using the representation. A result of the performance of the molecular-dynamics simulation includes a set of polymer conformations as a function of time. Each polymer conformation of the set of polymer conformations identifies, for each atom in the polymer, a position of the atom. For each polymer conformation of the set of polymer conformations, one or more spatial characteristics are determined, the spatial characteristics being of the polymer while in the polymer conformation. Each of the one or more spatial characteristics includes: a distance between two atoms (e.g., each of the two atoms being in a side chain of the one or more polymers or a polymer backbone chain of the polymer), an angle between three atoms in the polymer, or a dihedral angle of four atoms in the polymer backbone and the side-chain of the polymer. Based on the one or more spatial characteristics, an incomplete subset of the set of polymer conformations are determined. Each polymer conformation in the incomplete subset corresponds to an instance in which it is estimated that the polymer undergoes one or more reactions of a particular type. Based on a size of the incomplete subset, a probability of a reaction in which the polymer is a reactant and a particular other molecule is a product is estimated. The reaction probability can be output.

In various embodiments, a computer-implemented method is provided that comprises generating a representation of a polymer having one or more side chains; and performing a molecular-dynamics simulation using the representation. A result of the performance of the molecular-dynamics simulation includes a set of polymer conformations, each polymer conformation of the set of polymer conformations identifying, for each atom in the polymer, a position of the atom. The computer-implemented method further comprises determining, for each polymer conformation of the set of polymer conformations, one or more spatial characteristics of the polymer while in the polymer conformation. Each of the one or more spatial characteristics includes: a distance between two atoms, each of the two atoms being in a side chain of the one or more polymers or a polymer backbone chain of the polymer; an angle between three atoms in the polymer; or a dihedral angle of four atoms in the polymer backbone and the side-chain of the polymer. The computer-implemented method further comprises identifying, based on the one or more spatial characteristics, an incomplete subset of the set of polymer conformations estimated to undergo one or more reactions of a particular type; estimating, based on a size of the incomplete subset, a probability of a reaction in which the polymer is a reactant and a particular other molecule is a product; and outputting the reaction probability.

In some embodiments, the identification of the incomplete subset includes: identifying a distance criterion that, when satisfied, indicates that a nitrogen atom within the polymer backbone chain is within a predefined distance from a γ-carbon of the side chain; and determining that the distance criterion is satisfied for each side-chain conformation in the incomplete subset. The predefined distance may be less than or equal to 2.5 angstroms.

In some embodiments, the one or more reactions of the particular type includes a deamidation.

In some embodiments, the one or more reactions of the particular type includes an isomerization.

In some embodiments, the one or more spatial characteristics include multiple inter-atom distances, multiple angles, and/or multiple dihedral angles.

In some embodiments, the identification of the incomplete subset includes: identifying an acidity constraint that, when satisfied, indicates that a backbone amide of the polymer backbone chain is acidic, where the acidity constraint is configured to be satisfied when each of at least one backbone dihedral angle of the polymer is within a predefined corresponding range, the one or more spatial characteristics including the at least one backbone dihedral angle; and determining that the constraint criterion is satisfied for each polymer conformation in the incomplete subset.

In some embodiments, the at least one backbone dihedral angle includes a w dihedral angle and a ϕ dihedral angle of a reactant amino acid of the polymer and another amino acid of the polymer that is adjacent to the reactant amino acid.

In some embodiments, the identification of the incomplete subset includes: identifying an accessibility constraint that, when satisfied, indicates that an amide group of the polymer has above-threshold spatial accessibility to bind with a water molecule from a surrounding solvent; and determining, for each polymer conformation in the incomplete subset, that the accessibility constraint is satisfied based on assessing one or more geometrical characteristics of an intermediate molecule produced when an initial reaction occurs in which the polymer in the polymer conformation is a reactant.

In some embodiments, determining, for each polymer conformation in the incomplete subset, that the accessibility constraint is satisfied includes: executing a solvent-inclusive molecular dynamics simulation to simulate the polymer having the polymer conformation in a solvent; determining, based on one or more results of the execution of the solvent-inclusive molecular dynamics simulation, a water-blocking metric for the polymer conformation; and determining that the water-blocking metric is within a pre-defined open or closed range of values.

In some embodiments, the water-blocking metric is based on a number of frames that the amide group of the polymer binds with a water molecule in the solvent-inclusive molecular dynamics simulation.

In some embodiments, the computer-implemented method further comprises determining, based on the reaction probability, to include the polymer in a screen to assess binding affinity for a given target; and facilitating performance of the screen including the polymer.

In some embodiments, the polymer is an antibody or a polypeptide molecule.

In some embodiments, the computer-implemented method further comprises facilitating development of a liquid solution comprising the polymer as at least part of a therapeutic agent.

In some embodiments, the computer-implemented method further comprises, based on the predicted property of the solution: (i) adding the polymer to a list of potential polymers to be used as at least part of a therapeutic agent, (ii) removing the polymer from the list of potential polymers to be used as at least part of the therapeutic agent, (iii) ranking the polymer within the list of potential polymers to be used as at least part of the therapeutic agent, or (iv) a combination thereof.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 shows a representation of exemplary reactions, which can produce a chemically degraded product.

FIG. 2. illustrates exemplary conformation probabilities for side chains of aspartate.

FIGS. 3A-3B show exemplary simulated prevalence of various dihedral angles of aspartate side chains and particular dihedral angle ranges corresponding to reactive conformations.

FIGS. 4A-4B show exemplary data as to how isomerization and deamidation incidence depend on side-chain conformations.

FIG. 5 shows how an acidity of a molecule depends on the molecule's dihedral angles for N-formyl glycinamide.

FIGS. 6A-6C shows how an acidity of a molecule depends on the molecule's dihedral angles for Asn-Ser, Asn-Ala and Asn-Phe motifs.

FIG. 7 shows exemplary simulated prevalence of various backbone dihedral angle ranges of the amino acid next to the aspartate amino-acid (n+1 neighbor in the sequence) side chains and particular dihedral angle ranges corresponding to reactive conformations.

FIGS. 8A-8B show exemplary data as to how isomerization and deamidation incidence depend on an acidity of a backbone amide of a molecule.

FIG. 9 shows exemplary data as to how isomerization incidence depends on accessibility of a solvent.

FIG. 10 illustrates a process for generating a probability of a type of reaction based on a molecular-dynamic simulation and assessment of molecular spatial properties.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

A reaction involving a polymer (e.g., a polypeptide) that would produce a particular potential degraded product can include a reaction between multiple atoms of the polymer (e.g., a nucleophilic attack of nitrogen of a backbone amide on the γ-carbon of the side-chain). Whether the atoms react can depend on a physical proximity of the atoms and charges local to each atom, as well as environment conditions (e.g., access to a water molecule). Thus, for each of a set of conformations identified based on a molecular-dynamics simulation, multiple spatial characteristics of the polymer can be identified and used to predict a probability that the polymer will react to produce the particular degraded product.

I.A. Inter-Atom Distance Reaction Constraint

Whether a reaction between two atoms of a molecule (e.g., a nucleophilic attack on one of the two atoms) occurs can depend on a proximity of the two atoms. In some instances, spatial characteristics of a peptide conformation can include absolute or relative atom positions and/or the distance between two atoms. In some instances, spatial characteristics can include other geometry-associated information that can influence or determine how close two atoms in a molecule are to each other (and thus whether a reaction can occur), such as an angle between three atoms or a dihedral angle pertaining to some or all the atoms involved in the reaction (e.g., ψ and ϕ backbone dihedral angles of the amino-acids neighboring the isomerization or deamidation site). For example, the spatial characteristics can include two dihedral angles defined by eight atoms (Cₙ-Cαₙ-Cβₙ-Cγₙ and Nₙ-Cαₙ-Cₙ-Nₙ₊₁, where n corresponds to the aspartate or asparagine amino acids, and n+1 corresponds to the neighboring amino acid in the sequence), which can be used to estimate a distance between a backbone nitrogen atom and a γ-carbon of the side-chain group. The dihedral angles can be estimated by defining a space that corresponds to one dihedral angle (e.g., ψ) along one axis of the space and another dihedral angle (e.g., χ) along another axis of the space. Multiple regions within the space may be defined based on spatial characteristics, with each region being associated with a predicted reaction probability that may include a numerical probability, a categorical probability (e.g., very low, low, moderate, high) or a binary probability. For example, a first region can correspond to particular ranges of the dihedral angles (e.g., ψ and χ) that would configure the polymer such that a distance between two atoms that may participate in a nucleophilic attack is below a threshold (e.g., 2 angstroms or 3 angstroms). Meanwhile, a second (e.g., remaining) region can correspond to particular ranges of the dihedral angles that would configure the polymer such that the two atoms are separated by more than the threshold and thus unlikely to participate in a nucleophilic attack.

I.B. Acidity Constraint

Spatial proximity is one factor that influences whether a nucleophilic attack will occur. Other chemical properties of the polymer can also be influential. For example, a molecule with an acidic backbone amide may be more likely to participate in the reaction. Geometric conformations of the molecule can influence the molecule's chemical properties. For example, angles and/or dihedral angles can be indicative of chemical properties of the molecule, such as: an acidity of a backbone amide, and/or a propensity of an amino acid to act as a proton (H+) donor. A region (corresponding to a reaction probability) can thus be defined via one or more angle ranges so as to indicate a chemical property (e.g., backbone amide is sufficiently acidic) to predict the degradation reaction, in combination with aforementioned structural conformation.

It will be appreciated that regions may be separately defined to represent satisfaction/dissatisfaction of distance constraints and to represent satisfaction/dissatisfaction of acidity constraints (e.g., such that a first region is defined to indicate geometrical characteristics that correspond to particular atoms being separated by less than a threshold distance and that a second region is defined to indicate geometrical characteristics that correspond to a molecule having a reaction-friendly chemical property). Alternatively or additionally, one or more regions may be defined to collectively represent satisfaction/dissatisfaction of distance constraints and chemical-property constraints (e.g., such that a single region is defined to indicate geometrical characteristics that correspond to particular atoms being separated by less than a threshold distance and also that correspond to a molecule having a reaction-friendly chemical property).

I.C. Solvent Accessibility Reaction Constraint

Even if the inter-atom distance criterion is satisfied (e.g., based on an assessment of dihedral angles of the backbone and side-chain) and if the acidity criterion is satisfied (e.g., based on an assessment of the backbone dihedral angle for the neighboring amino-acid), chemical degradation does not occur without a solvent. Thus, an additional chemical-degradation constraint can require that a water molecule be accessible for hydrolysis. A constraint may be implemented by tracking a quantity of water molecules throughout an experiment. Thus, the experiment may track positions of each of multiple solvent molecules (e.g., and potentially each atom of each of multiple solvent molecules) in addition to tracking positions of individual atoms of the polymer. As each time step, it can be determined whether a solvent molecule is within a predefined distance from a particular site on the polymer (e.g., a backbone amide site of the polymer molecule). Some conformations may inhibit solvent molecules from accessing the particular polymer sites as a result of (for example) folds within the polymer. Alternatively, solvent accessibility surface area (SASA) of the side-chain and backbone amide can be calculated using geometry-based numerical methods (e.g. rolling a ball along the molecular surface). These SASA calculation methods can estimate the probability of finding the solvent molecules around the reaction sites, without explicitly simulating the water molecules around the polymer.

I.D. Experiment and Constraint Usage

Chemical degradation can involve sub-atomic interactions, covalent-bond formation and covalent-bond breakage. The experiment of these types of events using molecular dynamics is still impractical. Some techniques have predicted a reaction probability based on which amino acid motifs are present in a molecule. While reaction probabilities can differ dramatically across motifs, a motif's impact can depend on its location within a molecule (e.g., as to whether the motif is on a heavy chain or light chain and its position within a chain). Even for motifs that are considered highly stable, experimental data identifies some rare cases in which a reaction occurs at the motif despite the relative general stability.

In some instances, an experiment (e.g., a molecular-dynamics simulation and molecular-geometry technique) is performed to generate reaction probabilities. One or more iterations of the molecular-dynamics simulation can simulate how a polymer's conformation changes in time. A reaction probability can be generated for each of multiple conformations based on spatial characteristics (e.g., which can determine whether various reaction constraints are satisfied). For example, with respect to each conformation generated by a molecular dynamics simulation, spatial characteristics of the polymer in the conformation can be used to determine whether the inter-atom distance reaction constraint and the acidity reaction constraint is satisfied, which may then indicate the polymer having the conformation would be ripe for participation in a reaction. The experiment(s) can be performed using a molecular simulation ensemble that identifies experimental parameters of the system that are to be fixed (e.g., a combination of two or more of: particle numbers (N), volume (V), energy (E), sum of kinetic energy, potential energy, temperature (T), and pressure (P)). For example, an ensemble can include NVE, NVT, or NPT. The experiment(s) can use an integrator to integrate an equation of motion and a thermostat or barostat to control temperature ore pressure throughout the experiment. The experiment(s) may be performed for a particular number of time steps or until a target equilibration is reached.

Solvent-inclusive modeling can be used to estimate a proportion of the polymers molecules favorably configured for reaction that have access to and react with a solvent molecule to produce a particular degraded product. Based on a fraction of the experiment-generated polymer conformations for which each constraint is satisfied, an output can be generated that indicates whether, an extent to which and/or a speed at which a given polymer chemically degrades to the particular degraded product. (It will be appreciated that the experiment may generated multiple outputs of a same conformation or having same spatial properties, which can be uniquely considered.) Thus, experiment-based techniques disclosed herein can generate predicted reaction susceptibility based on molecular dynamics and analyses of three-dimensional structures of various conformations of a polymer (e.g., rather than on conformation-independent data corresponding to identities of amino groups in the polymer).

### II. Definitions

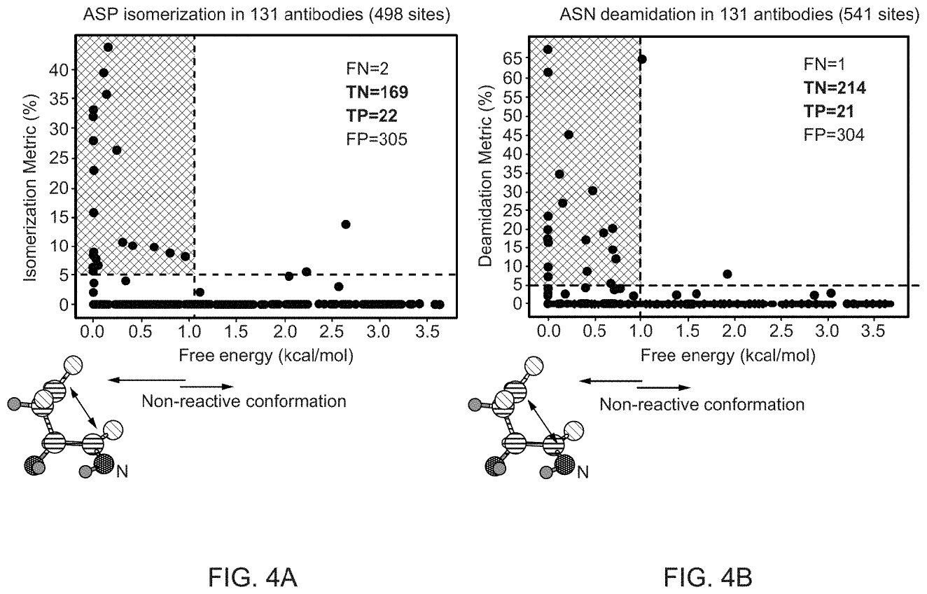

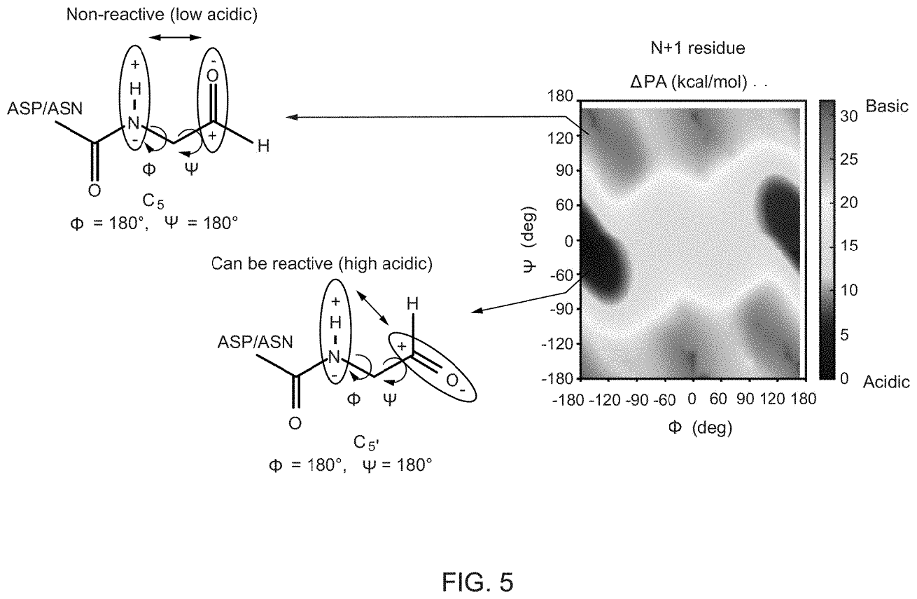

The term “polymer”, as used herein, is used to refer to a molecule that includes multiple molecules that are connected via bonds. A polymer can include a polypeptide that includes multiple amino acids. A polymer can be or can include a protein, an antibody, an oligosaccharide, DNA and/or RNA. Amino acids within the polymer can be linked together via peptide bonds. The polymer can include a protein including any protein modality, such as an amino acid substituted (un-natural amino acid), alternate glycation, protein, DNA complex and/or virus surface-coat protein. The polymer may be linear or branched, it may comprise modified amino acids, and it may be interrupted by non-amino acids. The polymer may include backbone that includes a first set of amino acids and one or more side chains (each including a second set of amino acids). The term also encompasses an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Further, a polypeptide can include an antibody and/or antibiotic polypeptide, such as antibodies referenced below in relation to FIGS. 4A-4B.

The term “conformation”, as used herein in relation to a polymer, peptide or polypeptide, is used to refer to a spatial arrangement of atoms. The conformation of a polypeptide can characterize a conformation of a backbone of the polypeptide as well as a conformation of each side chain of the polypeptide.

The term “representation of a polymer”, as used herein, is used to refer to an identification of compositional and spatial characteristics of the polymer. For example, the representation can identify which atoms are included in the polymer, which atoms are in a backbone of the polymer, which atoms are in individual sidechains of the polymer, a chemical formula of the polymer and/or a chemical name of the polymer.

The term “spatial characteristic of a polymer”, as used herein, is used to refer to information that indicates positions of one or more first parts of the polymer relative to one or more second parts of the polymer. For example, a spatial characteristic can include a distance between two atoms, an angle between three atoms or an angle between four atoms of the polymer.

The term “experiment”, as used herein, is used to refer to a computational experiment that can predict whether and/or how a structure of a molecule may change in time and/or in a presence of a solvent. An experiment can include (for example) a simulation (e.g., a molecular dynamics simulation) or an artificial-intelligence approach.

The term “chemical degradation”, as used herein, is used to refer to a process by which a molecule (e.g., a polypeptide molecule or polymer molecule) is broken down into two or more fragments. In the context of a polymer, chemical degradation can include a full depolymerization of the polymer to corresponding monomers or a partial depolymerization (e.g., to one or more oligomers and potentially one or more other chemical substances). Chemical degradation can include a particular type of chemical process, such as tryptophan oxidation, methionine oxidation, ASN-PRO clipping, asparagine deamidation, aspartate isomerization, or any other chemical reaction that can result in altering the chemical behavior of the aforementioned polymer.

### III. Exemplary Dependency of Reaction Occurrence

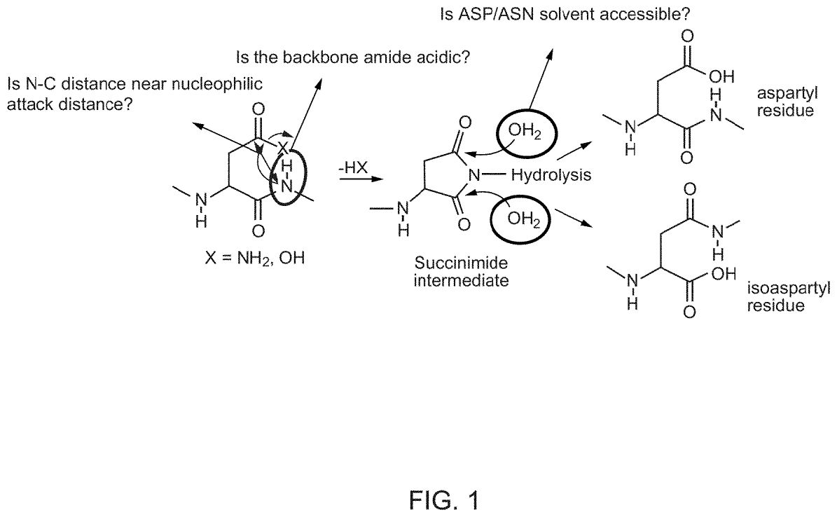

FIG. 1 shows a representation of exemplary reactions, which can produce a chemically degraded product. More specifically, FIG. 1 depicts a representation of a side chain with a backbone amide group. If the backbone amide group is sufficiently acidic and if the backbone nitrogen atom and the γ-carbon of the side-chain group are in sufficiently close proximity, a nucleophilic attack on the backbone amine group (the α-amino group of the C-terminally flanking amino acid). A metastable succinimide (cyclic imide) intermediate can be produced as a result of the nucleophilic attack. If a solvent is accessible to the succinimide intermediate, the succinimide hydrolyzes to a mixture of aspartyl and iso-aspartyl linkages. Alternatively, nucleophilic attack by the backbone carbonyl oxygen results in a cyclic isoimide intermediate, yielding only aspartyl residues after hydrolysis independent of the point of attack of the incoming water molecule. Asparagine residues can deamidate to Asp by direct water-assisted hydrolysis.

With respect to an aspartyl residue, the polypeptide may maintain its target characteristics. However, with respect to an isoaspartyl residue, a conformation of the protein and its electrostatic properties can be changed relative to the original polypeptide. If an experiment can reliably predict a probability that a polypeptide will chemically degrade to an undesired product, polypeptides and/or formulations may be selected accordingly to minimize the undesired chemical degradation and maintain an active polypeptide having a target functionality.

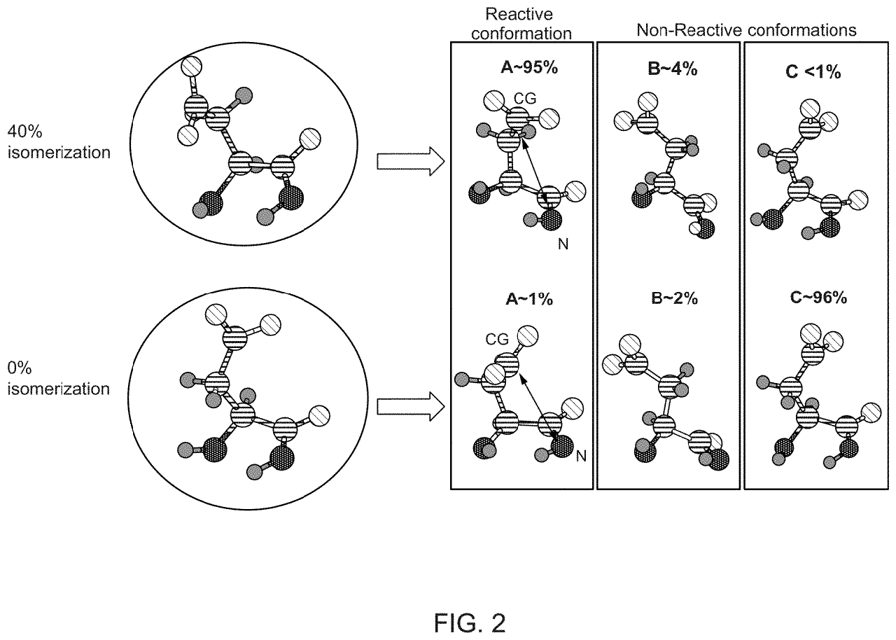

FIG. 2. illustrates exemplary conformation probabilities for side chains of aspartate. The two rows correspond to the initial conformation for aspartate amino acid across two different antibody fab structures with high and low experimentally measured reaction rates (40% and 0% shown on the left side). The water molecules and other amino acids in the two fab structures were simulated but are not shown. The right sides show the probabilities that the initial conformation will transition into a reactive conformation (where a nitrogen of a backbone amide is within two angstroms from a γ-carbon of the side chain) or one of two non-reactive conformations. These three conformations were identified by performing a cluster analysis on molecular-dynamics trajectories to identify the three most representative side-chain conformations for aspartate. As shown, these three conformations correspond to about 99% of the conformations observed throughout the experiment. Notably, the probability distributions are very different across the two aspartate amino-acids, and the calculated probability of reactive conformations (e.g. 95% and 1%) are in accordance with the measured isomerization rates for the two aspartate amino-acids (e.g. 40% and 0%).

### IV. Inter-Atom Distance Constraint Implementation

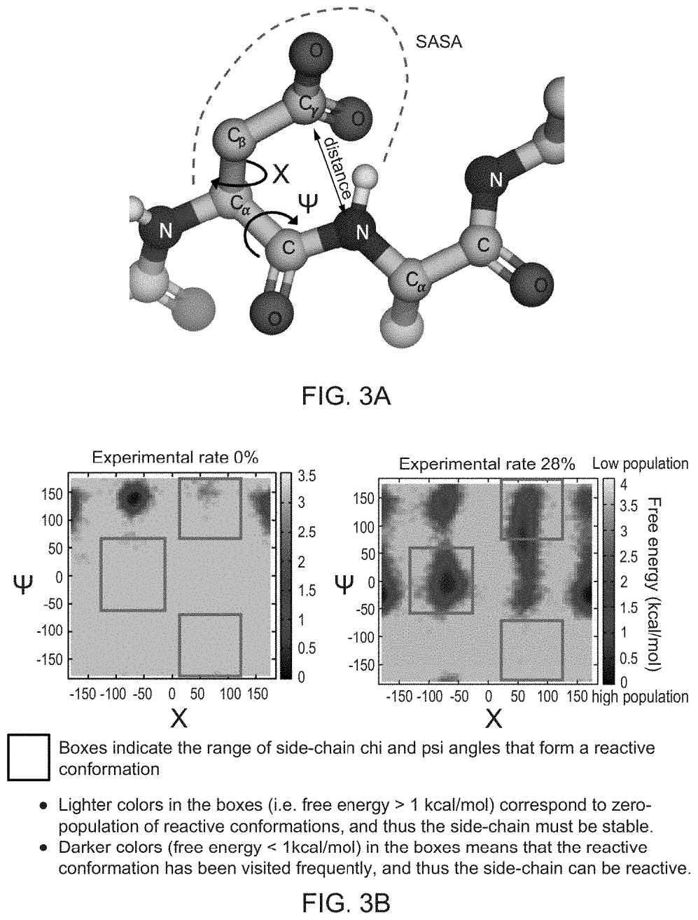

FIGS. 3A-3B show exemplary simulated prevalence of various dihedral angles of aspartate side chains and particular dihedral angle ranges corresponding to reactive conformations. More specifically, whether a given atom will attack or react with another atom depends on their proximity. In some instances, atoms' positions are tracked throughout an experiment, and thus, the distance can also be tracked. In other instances, dihedral angles can be tracked throughout the experiment, which can be used to infer or estimate whether the atoms are sufficiently close to react. FIG. 3A shows how two dihedral angles, X and Ψ, affect a distance between a backbone nitrogen atom and a γ-carbon of the side-chain group.

FIG. 3B show two graphs representing, across a range of X values and a range of Ψ values, a free energy of a conformation associated with those angles. The free energy values can be generated via a molecular-dynamics model. Lighter colors in the boxes (i.e. free energy >1 kcal/mol) correspond to zero-population of reactive conformations, and thus the side-chain must be stable. Darker colors (free energy <1 kcal/mol) in the boxes means that the reactive conformation has been visited frequently, and thus the side-chain can be reactive.

The boxes identify particular dihedral-angle ranges that, geometrically, position the nitrogen of a backbone amide and the γ-carbon of the side-chain within covalent bond distance (˜2 angstroms). If the boxed regions do not include conformations associated with low free energy values, the outputs indicate that the polymer is unlikely to chemically degrade as a result of conformations of the polymer molecule not bringing the backbone nitrogen atom sufficiently close to the γ-carbon of the side-chain group to react.

Each of the left plot and the right plot corresponds to an experiment performed using a particular polymer structure. Notably, the right plot indicates that the corresponding polymer is likely to have conformations in which a distance between the nitrogen of the backbone amide and the γ-carbon of the side-chain within the threshold distance. Meanwhile, the polymer corresponding to the left plot is unlikely to be in conformations for which the atoms are in this proximity. Any atom-explicit or atom-implicit solvent molecular dynamics simulation can be performed, and the simulation can be of the fab structure, fv or full-length antibody.

For this particular case, all atom-explicit solvent molecular dynamics of the fab structure were performed. Specifically, the GPU implementation of Amber 2015 molecular dynamics (MD) software package3 with the SPFP precision model8 was used for the MD simulation using the following protocol. The structure was relaxed with 2,000 steps of conjugate-gradient energy minimization, using harmonic restraining potential with the force constant of 10 kcal/mol/Å² to restrain the solute to the initial structure with solute atoms restrained to the initial structure by a harmonic positional restraint of strength 10 kcal/mol/Å². Then the pressure was maintained at 1 atmosphere and the thermostat temperature increased to 300 K over the course of 200 ps, while applying Harmonic positional restraints of strength 10 kcal/mol/Å² to the protein structure. The system was then equilibrated for 1 ns with a restraint force constant of 1 kcal/mol/Å2. All restraints were removed for the production stage. The simulation time step was 4 fs. A 9 Å cutoff radius was used for range limited interactions, with Particle Mesh Ewald electrostatics for long-range interactions. The production simulation was carried out using NPT conditions. Langevin dynamics9 was used to maintain the temperature at 300 K with a collision frequency of 1 ps-1. The production stage of the MD simulation was performed for 500 ns. During dynamics the SHAKE algorithm10 was applied to constrain all bonds involving hydrogen atoms. Snapshots from the MD trajectory were saved every 10 ps for analysis. Default values were used for all other simulation parameters. The protocol described above was repeated to generate 3 independent replicates of 500 ns trajectories, adding up to 1.5 microsecond trajectories for each structure.

FIGS. 4A-4B show exemplary data as to how isomerization and deamidation incidence depend on side-chain conformations. For all Aspartate and Aspargine residues within the CDR loops of a set of 131 antibodies (as identified in Lu et al. (2019), “Deamidation and isomerization liability analysis of 131 clinical-stage antibodies”, mAbs, 11:1, 45-57, which is hereby incorporated by reference in its entirety for all purposes), the reaction rates experimentally measured under standardized conditions are plotted against a free-energy value in the regions of X and Ψ 2-dimensional free energy plot that correspond to a conformation for which the nitrogen of a backbone amide and the γ-carbon of the side-chain are within covalent bond distance (e.g., corresponding to a minimum of the free-energy values across pixels in the boxes shown in FIG. 3B).

For each of the antibodies, isomerization and deamidation reccation rates for all of the aspartate and asparagine amino acids in the DCR loops were experimentally determined, which indicates the fraction of aspartate or asparagine sites that underwent isomerization or deamidation (respectively) to produce a chemically degraded product. FIGS. 4A and 4B compare the isomerization metric and deamidation metric (respectively) to the minimum free energy in the free-energy surface region that corresponds to the reactive conformations (i.e., boxes shown in FIG. 3B). A free-energy threshold was set (G=1) to identify a free energy value indicating that a polymer was likely to have conformations with inter-atom distances sufficiently close for a reaction to occur. A modification threshold was also set at 5% to indicate that a substantial degree of isomerization or deamidation occurred. (Another threshold value could instead be set.)

Results were deemed to be accurate when (1) a minimum free energy was below the free-energy threshold and a modification metric was above the modification threshold; or (2) a minimum free energy was above the free-energy threshold and a modification metric was below the modification threshold. Notably, there were many true negatives. More specifically, of all polymers for which a minimum free-energy value was greater than the free-energy threshold, the isomerization metric was below the modification threshold for 98.8% of these instances. Of all polymers for which a minimum free-energy value was greater than the free-energy threshold, the deamidation metric was below the modification threshold for 99.5% of these instances. Thus, high minimum free energy values (meaning that a polymer was unlikely to have a conformation where nitrogen of a backbone amide and the γ-carbon of the side-chain are in close proximity) are highly predictive of a lack of isomerization and deamidation occurrences.

### V. Acidity Constraint Implementation

FIG. 5 shows how an acidity of a molecule depends on the molecule's dihedral angles for N-formyl glycinamide representing a simplified structure of Asn-Gly motif. The molecular-conformation representations in FIG. 5 show how acidity of a backbone amide can depend on the n+1 residue backbone dihedral angles when n+1 does not have a side-chain (e.g., glycine). When the dihedral angles oppositely align the local electrostatic dipoles produced by the backbone amide and carbonyl groups (i.e., NH and CO groups) (to thus cause low backbone amide acidity), the opposite polarities can provide stability, such that a nucleophilic attack is unlikely to occur. Meanwhile, when the dihedral angles are configured to instead align two local dipoles produced by the backbone amide and carbonyl groups (i.e., NH and CO groups) (to thus cause high backbone amide acidity), the same polarities can provide instability, which can trigger a nucleophilic attack.

The heat map shows how acidity depends on the dihedral angles Φ and Ψ. More specifically, the heat map shows proton affinity of the hydrogen on the amide group based on the dihedral angles. In the absence of n+1 residue side-chain (as in glycine), acidity is more highly dependent on the Ψ angle, as compared to the Φ angle.

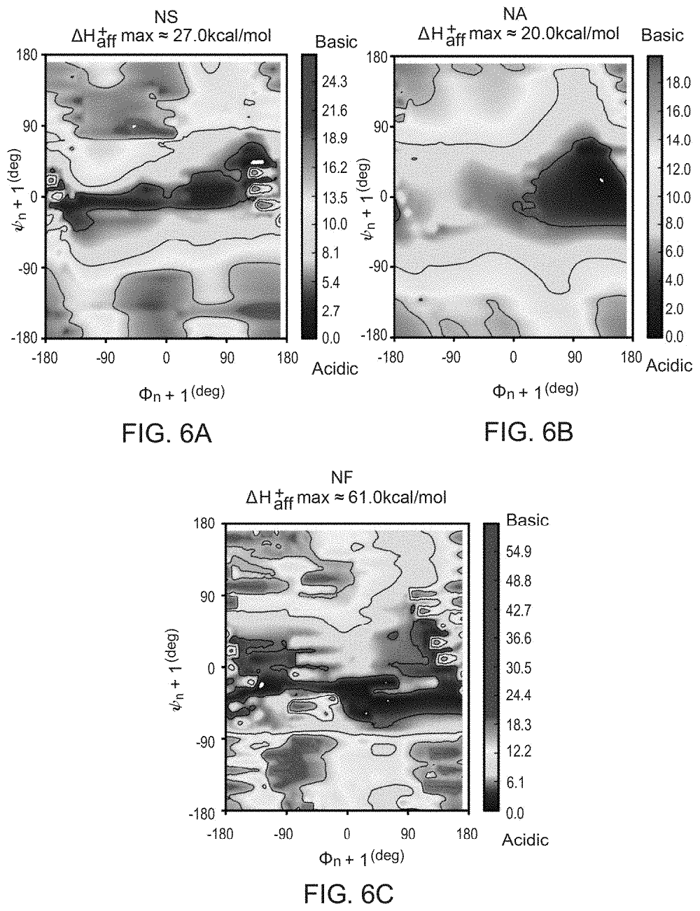

As noted, FIG. 5 pertains to the Asn-Gly motif. FIGS. 6A-6C show how an acidity of a molecule depends on the molecule's dihedral angles for a few other motifs. More specifically, FIG. 6A, FIG. 6B and FIG. 6C correspond to Asn-Ser (NS), Asn-Ala (NA) and Asn-Phe (NF) motifs, respectively. Across all of these motifs (and the Asn-Gly motif), high acidity is strongly correlated with the W angle and is associated with small W angles. Acidity can correlate with the 0 angle as well, but the extent of the correlation depends on the type of n+1 amino-acid side-chain. For example, in Asn-Ala motif, for the small values of the W angle, the acidity is higher when the 0 angle is positive.

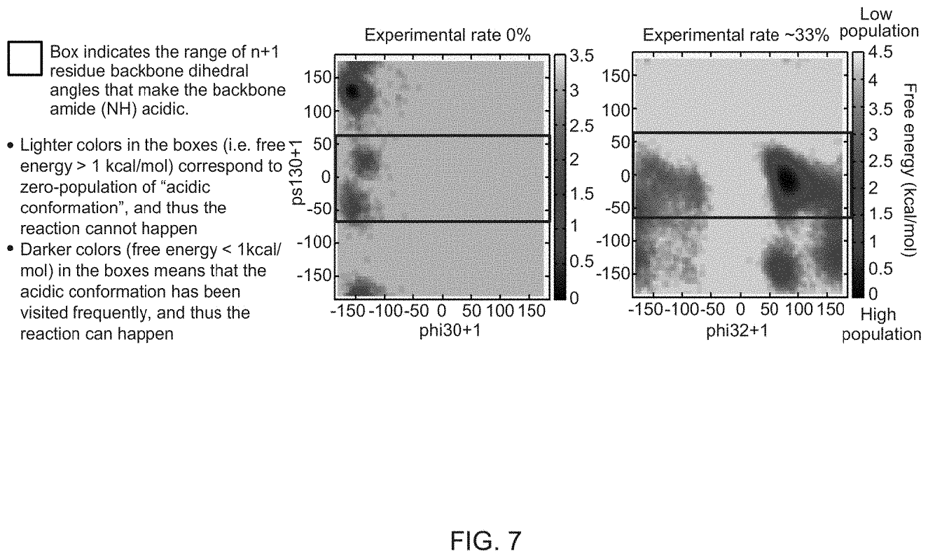

FIG. 7 shows exemplary simulated prevalence of various backbone dihedral angle ranges of the amino acid next to the aspartate amino acid (n+1 neighbor in the sequence) and particular dihedral angle ranges corresponding to reactive conformations. Each heat map shows, across a range of Φ values and a range of Ψ values, a free energy of a conformation associated with those angles. The free energy values can be calculated from molecular-dynamics simulations. Lighter colors in the boxes (i.e. free energy >1 kcal/mol) correspond to zero-population of “acidic conformation”, and thus the reaction cannot happen. Darker colors (free energy <1 kcal/mol) in the boxes means that the acidic conformation has been visited frequently, and thus the reaction can happen.

The box identifies particular dihedral-angle ranges that make the backbone amide acidic. If the boxed region does not include conformations associated with low free energy values, the outputs indicate that the polymer is unlikely to chemically degrade as a result of conformations of the polymer molecule being stable and thus unlikely to undergo a nucleophilic attack.

Each of the left plot and the right plot corresponds to a simulation using a particular polymer structure. Notably, the right plot indicates that the corresponding polymer is more likely to have acidic conformations. Thus, the polymer corresponding to the left plot is more stable and less likely to chemically degrade.

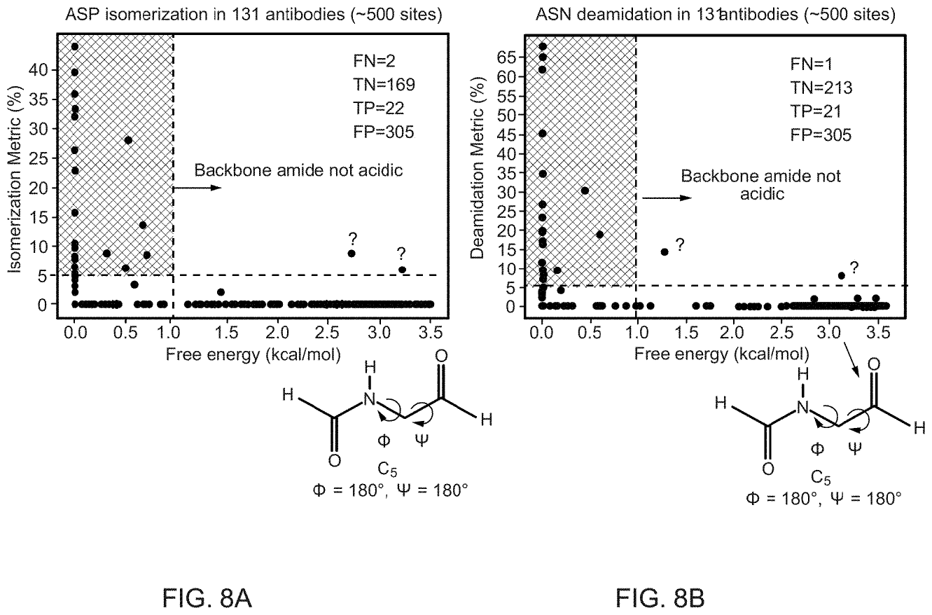

FIGS. 8A-8B show exemplary data as to how isomerization and deamidation incidence depend on an acidity of a backbone amide of a molecule. For each of a set of commercially available antibodies (e.g., those represented in FIGS. 4A-4B), a minimum free-energy value that corresponds to a minimum free energy value across each pair-wise combination of 0 and W values that are within the region shown (via the box) in FIG. 7.

For each of the antibodies, an isomerization metric and a deamidation metric were experimentally determined, as described with respect to FIGS. 4A-4B. FIGS. 8A and 8B compare the isomerization metric and deamidation metric (respectively) to the minimum free energy. A free-energy threshold and a modification threshold were set, as described with respect to FIGS. 4A-4B.

Results were deemed to be accurate when (1) a minimum free energy was below the free-energy threshold and a modification metric was above the modification threshold; or (2) a minimum free energy was above the free-energy threshold and a modification metric was below the modification threshold. There were many true negatives. More specifically, of all polymers for which a minimum free-energy value was greater than the free-energy threshold, the isomerization metric was below the modification threshold for 98.8% of these instances. Of all polymers for which a minimum free-energy value was greater than the free-energy threshold, the deamidation metric was below the modification threshold for 99.1% of these instances. Thus, high minimum free energy values (indicating that a polymer is unlikely to have a highly acidic conformation) are highly predictive of a lack of isomerization and deamidation occurrences. Notably, the simulation-based approach to predict whether a molecule will be acidic and thus prone to chemical degradation can provide useful results even for large molecules, such as proteins.

### VI. Accessibility Constraint Implementation

Even if a nucleophilic attack occurs, a polymer is not degraded unless a water molecule is accessible to the amide group. A molecular dynamics simulation can be configured to simulate the polymer in a solvent (e.g., as an explicit solvent or implicit solvent). A water-blocking metric can be defined as a number of frames that the amide group binds with a non-water group minus a number of frames that the amide group binds with a water molecule. Thus, the metric indicates frames of solvent accessibility. Thus, negative metrics correspond to greater water accessibility as compared to positive metrics. Positive metrics may indicate that a geometry of a polymer blocks a water molecule from reaching the amide group.

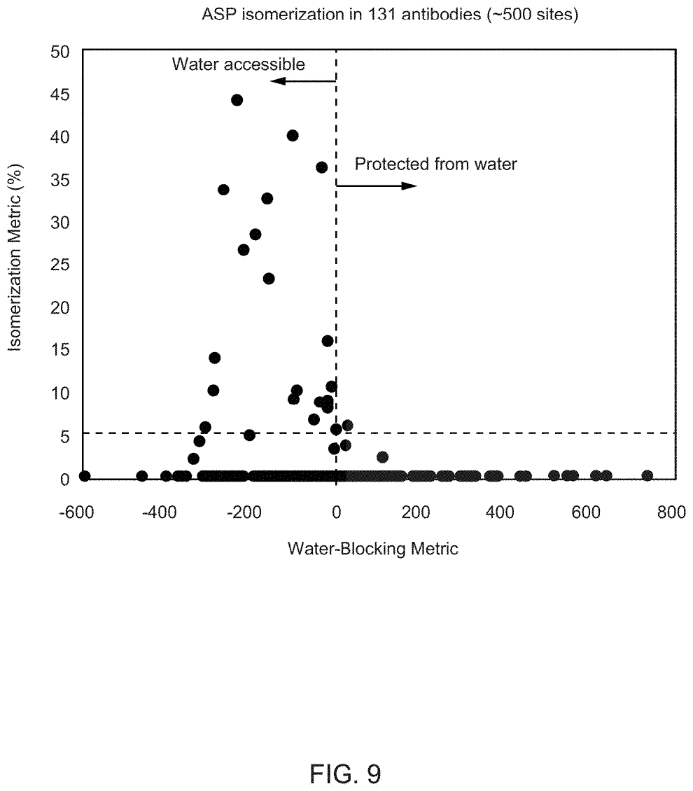

FIG. 9 shows exemplary data as to how isomerization incidence depends on accessibility of a solvent. More specifically, a solvent-inclusive molecular dynamics simulation was run for each of 131 polypeptides. The experimentally observed isomerization modification percentages is plotted against the water-blocking metrics for the 131 polypeptides. A water-blocking threshold for the water-blocking metric was set at 0.

Only one false negative for which the water-blocking metric was above the water-blocking threshold and for which the modification metric was above the modification threshold was observed. Thus, positive water-blocking values (indicating a lack of amide-group access to water) are highly predictive of a lack of isomerizations.

### VII. Process for Predicting Reaction Type for Polymer

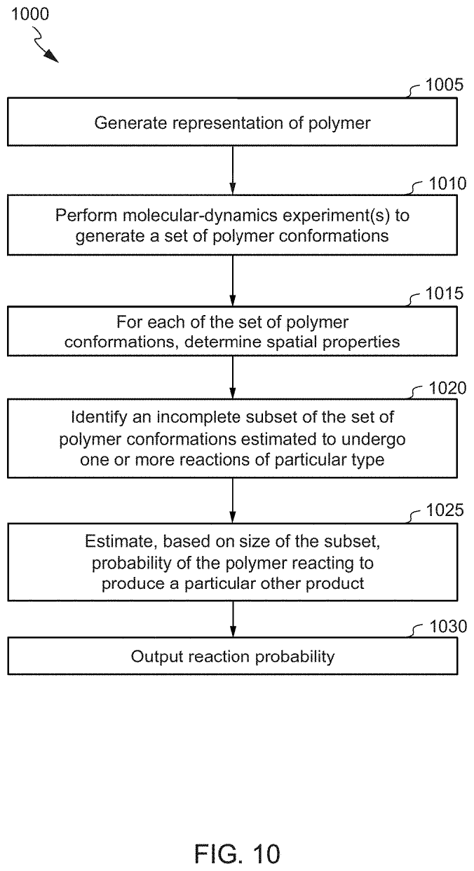

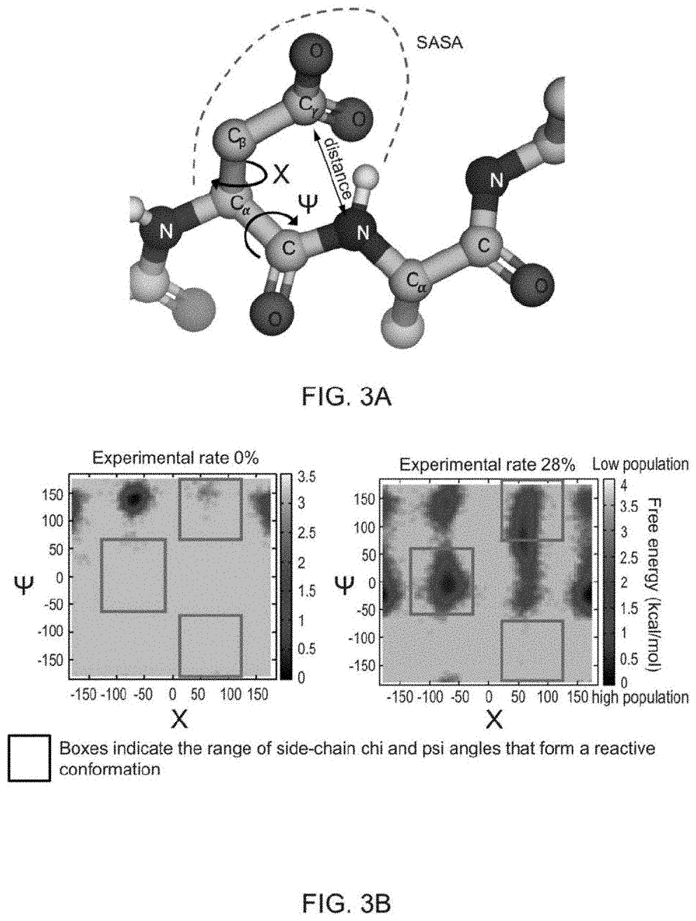

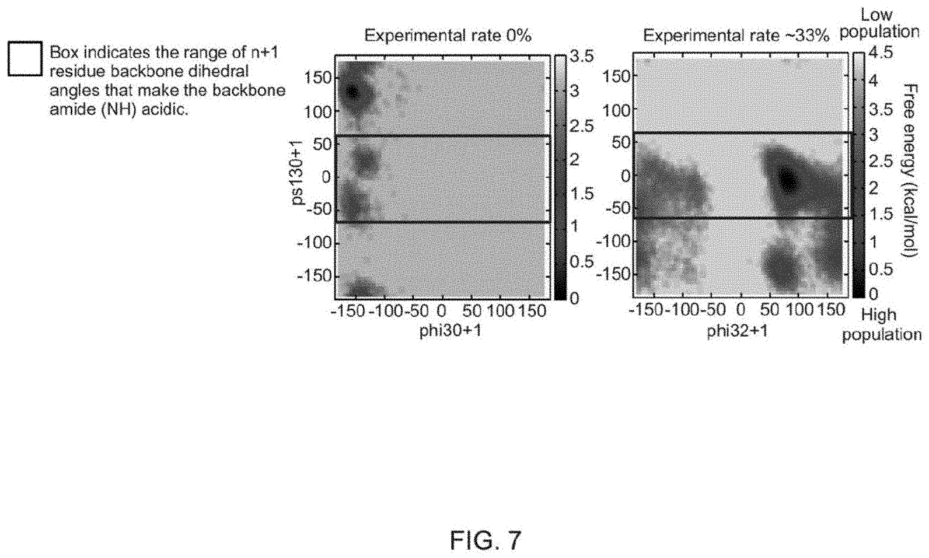

FIG. 10 illustrates a process 1000 for generating a probability of a type of reaction based on a molecular-dynamic experiment and assessment of molecular spatial properties. Process 1000 begins at block 1005, where a representation of a polymer is generated. The representation can include an identification of atoms, masses, charges and inter-atom connections for a polymer (and potentially for a solvent). The representation can further include starting coordinates for each atom of the polymer (and potentially for the solvent). The representation may further include constraints to be computationally applied throughout the experiment, such as limits on angles or dihedrals, Van der Waals terms, etc.

At block 1010, one or more molecular-dynamics experiments are performed to generate a set of polymer conformations. Each polymer conformation of the set of polymer conformations can correspond to a time step in the experiment(s). Each polymer conformation of the set of polymer conformations can include, for each atom of the polymer, a position of the atom. The set of polymer conformations can be determined by calculating forces from particle positions and numerically solving equations of motion. At each time step, in addition to determining a position of each atom, a momenta of each atom can further be estimated.

At block 1015, for each of the set of polymer conformations, one or more spatial properties of the polymer can be determined based on the positions of the atoms indicated in the polymer conformation. The one or more spatial properties can include a distance between two atoms of the polymer (e.g., between a backbone nitrogen atom and a γ-carbon of a side-chain group and/or between two atoms that would need to be involved in a nucleophilic attack to produce a particular partly or fully degraded product). The one or more spatial properties can include an angle and/or dihedral angle (e.g., a ψ, ϕ and/or X backbone dihedral angle of amino-acids neighboring the isomerization or deamidation site).

At block 1020, an incomplete subset of the set of polymer conformations are identified. The incomplete subset is identified such that each polymer conformation in the set of polymer conformations is estimated to undergo one or more reactions of a particular type (e.g., a chemical-degradation reaction, such as isomerization or deamidation). The particular type may include a type of reaction that produces a particular other degraded product. The incomplete subset can be identified based on the spatial properties of the set of polymers. For example, one or more reaction constraints may be identified that indicate partial types of spatial properties that a polymer is to have that enable a reaction to occur. The one or more reaction constraints can include particular ranges of particular dihedral angles and/or particular distances between atoms.

In some instances, the molecular-dynamic experiment(s) further includes a solvent. A separation distance between a particular part of the polymer conformation and a solvent molecule (e.g., a closest solvent molecule) can also be estimated in correspondence with each of the set of polymer conformations. A reaction constraint may identify an upper threshold for the separation distance.

Thus, the incomplete subset can be identified to include polymer conformations for which each of one or more reaction constraints are satisfied. Evaluation of the reaction constraints can depend on relative positions of atoms, as determined based on atom-specific position data indicated in individual polymer conformations.

At block 1025, a probability of the polymer reacting to produce a particular other degraded product is estimated. The particular other degraded product can include one produced by a reaction of the particular type. The particular other degraded product can include a chemically degraded form of the polymer. The probability of the polymer reacting to produce the particular other degraded product can be estimated based on the size of the incomplete subset and potentially also the size of the set of polymers. In some instances, the probability is estimated to be a size of the incomplete subset relative to a size of the set of polymers.

At block 1030, the reaction probability is output. For example, the reaction probability is sent to a file, displayed on a screen, or emailed to a specified email address. In some instances, the reaction probability is used to select a polymer to be used in a particular manner (e.g., to develop a treatment for a particular condition) and/or to select a particular formulation for the polymer (e.g., to restrict water accessing the polymer). In some instances, the reaction probability is output to a screening system, such that the output can be used to select an incomplete subset of candidate molecules from a set of candidate molecules to include in a screen (e.g., a high-throughput screen). The screen may determine whether and/or an extent to which each screened candidate molecule binds to a given target (e.g., so as to identify a binding affinity) and/or is chemically stable. The reaction probability may be used (in pre- or post-manufacturing) during lead-candidate selection when no or little material is available.

The selection of the incomplete subset can be performed automatically (e.g., based on reaction probabilities associated with the set of candidate molecules and potentially one or more other metrics), semi-automatically and/or based on input. For example, the subset may defined to include a particular number of candidate molecules that are associated with the lowest reaction probabilities across the set of candidate molecules. As another example, an initial filtering can be performed to identify candidate molecules from the set of candidate molecules that have a reaction probability that is below an absolute or relative threshold, and the subset can be selected from the filtered set (e.g., via user input and/or one or more other metrics). As yet another example, an interface may be presented that identifies each of some or all of the set of candidate molecules along with associated reaction probabilities, and the interface can be configured to receive input that indicates which of the set of candidate molecules are to be included in the screen. Upon having identified the incomplete subset, an instruction may be transmitted that indicates that the candidate molecules in the incomplete subset are to be deposited into a screening unit (e.g., well, test tube, etc.).

In some instances, the process 1000 further includes comparing the reaction probability to a predetermined threshold; moving forward with manufacturing; selecting the polymer for further processing (e.g., alongside other factors such as clearance rate); and/or facilitating development of a liquid solution comprising the antibody molecule as at least part of a therapeutic agent. For example, the development of a liquid solution comprising the antibody molecule as at least part of a therapeutic agent may be facilitated based, at least partially, on the reaction probability being below or above the predetermined threshold. In some instances, the process 1000 further includes, based on the reaction probability of the polymer: (i) adding the polymer to a list of potential polymers to be used as at least part of a therapeutic agent, (ii) removing the polymer from the list of potential polymers to be used as at least part of the therapeutic agent, (iii) ranking the polymer within the list of potential polymers to be used as at least part of the therapeutic agent, or (iv) a combination thereof.

### VIII. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention.

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A computer-implemented method comprising:
generating a representation of a polymer having one or more side chains;
performing a molecular-dynamics simulation using the representation, wherein a result of the performance of the molecular-dynamics simulation includes a set of polymer conformations, each polymer conformation of the set of polymer conformations identifying, for each atom in the polymer, a position of the atom;
determining, for each polymer conformation of the set of polymer conformations, one or more spatial characteristics of the polymer while in the polymer conformation, wherein each of the one or more spatial characteristics includes:
a distance between two atoms, each of the two atoms being in a side chain of the one or more polymers or a polymer backbone chain of the polymer;
an angle between three atoms in the polymer; or
a dihedral angle of four atoms in the polymer backbone and the side-chain of the polymer;
identifying, based on the one or more spatial characteristics, an incomplete subset of the set of polymer conformations estimated to undergo one or more reactions of a particular type;
estimating, based on a size of the incomplete subset, a probability of a reaction in which the polymer is a reactant and a particular other molecule is a product; and
outputting the reaction probability.

2. The computer-implemented method of claim 1, wherein the identification of the incomplete subset includes:
identifying a distance criterion that, when satisfied, indicates that a nitrogen atom within the polymer backbone chain is within a predefined distance from a γ-carbon of the side chain; and
determining that the distance criterion is satisfied for each side-chain conformation in the incomplete subset.

3. The computer-implemented method of claim 1, wherein the identification of the incomplete subset includes:
identifying an acidity constraint that, when satisfied, indicates that a backbone amide of the polymer backbone chain is acidic, and wherein the acidity constraint is configured to be satisfied when each of at least one backbone dihedral angle of the polymer is within a predefined corresponding range, the one or more spatial characteristics including the at least one backbone dihedral angle; and
determining that the constraint criterion is satisfied for each polymer conformation in the incomplete subset.

4. The computer-implemented method of claim 1, wherein the identification of the incomplete subset includes:
identifying an accessibility constraint that, when satisfied, indicates that an amide group of the polymer has above-threshold spatial accessibility to bind with a water molecule from a surrounding solvent; and
determining, for each polymer conformation in the incomplete subset, that the accessibility constraint is satisfied based on assessing one or more geometrical characteristics of an intermediate molecule produced when an initial reaction occurs in which the polymer in the polymer conformation is a reactant.

5. The computer-implemented method of claim 4, wherein determining, for each polymer conformation in the incomplete subset, that the accessibility constraint is satisfied includes:
executing a solvent-inclusive molecular dynamics simulation to simulate the polymer having the polymer conformation in a solvent;
determining, based on one or more results of the execution of the solvent-inclusive molecular dynamics simulation, a water-blocking metric for the polymer conformation; and
determining that the water-blocking metric is within a pre-defined open or closed range of values.

6. The computer-implemented method of claim 1, further comprising:
determining, based on the reaction probability, to include the polymer in a screen to assess binding affinity for a given target; and
facilitating performance of the screen including the polymer.

7. The computer-implemented method of claim 1, further comprising, based on the reaction probability: (i) adding the polymer to a list of potential polymers to be used as at least part of a therapeutic agent, (ii) removing the polymer from the list of potential polymers to be used as at least part of the therapeutic agent, (iii) ranking the polymer within the list of potential polymers to be used as at least part of the therapeutic agent, or (iv) a combination thereof.

8. A system comprising:
one or more data processors; and
a non-transitory computer readable storage medium containing instructions which, when executed on the one or more data processors, cause the one or more data processors to perform actions including:
generating a representation of a polymer having one or more side chains;
performing a molecular-dynamics simulation using the representation, wherein a result of the performance of the molecular-dynamics simulation includes a set of polymer conformations, each polymer conformation of the set of polymer conformations identifying, for each atom in the polymer, a position of the atom;
determining, for each polymer conformation of the set of polymer conformations, one or more spatial characteristics of the polymer while in the polymer conformation, wherein each of the one or more spatial characteristics includes:
a distance between two atoms, each of the two atoms being in a side chain of the one or more polymers or a polymer backbone chain of the polymer;
an angle between three atoms in the polymer; or
a dihedral angle of four atoms in the polymer backbone and the side-chain of the polymer;
identifying, based on the one or more spatial characteristics, an incomplete subset of the set of polymer conformations estimated to undergo one or more reactions of a particular type;
estimating, based on a size of the incomplete subset, a probability of a reaction in which the polymer is a reactant and a particular other molecule is a product; and
outputting the reaction probability.

9. The system of claim 8, wherein the identification of the incomplete subset includes:
identifying a distance criterion that, when satisfied, indicates that a nitrogen atom within the polymer backbone chain is within a predefined distance from a γ-carbon of the side chain; and
determining that the distance criterion is satisfied for each side-chain conformation in the incomplete subset.

10. The system of claim 8, wherein the identification of the incomplete subset includes:
identifying an acidity constraint that, when satisfied, indicates that a backbone amide of the polymer backbone chain is acidic, and wherein the acidity constraint is configured to be satisfied when each of at least one backbone dihedral angle of the polymer is within a predefined corresponding range, the one or more spatial characteristics including the at least one backbone dihedral angle; and
determining that the constraint criterion is satisfied for each polymer conformation in the incomplete subset.

11. The system of claim 8, wherein the identification of the incomplete subset includes:
identifying an accessibility constraint that, when satisfied, indicates that an amide group of the polymer has above-threshold spatial accessibility to bind with a water molecule from a surrounding solvent; and
determining, for each polymer conformation in the incomplete subset, that the accessibility constraint is satisfied based on assessing one or more geometrical characteristics of an intermediate molecule produced when an initial reaction occurs in which the polymer in the polymer conformation is a reactant.

12. The system of claim 11, wherein determining, for each polymer conformation in the incomplete subset, that the accessibility constraint is satisfied includes:
executing a solvent-inclusive molecular dynamics simulation to simulate the polymer having the polymer conformation in a solvent;
determining, based on one or more results of the execution of the solvent-inclusive molecular dynamics simulation, a water-blocking metric for the polymer conformation; and
determining that the water-blocking metric is within a pre-defined open or closed range of values.

13. The system of claim 8, wherein the actions further include:
determining, based on the reaction probability, to include the polymer in a screen to assess binding affinity for a given target; and
facilitating performance of the screen including the polymer.

14. The system of claim 8, wherein the actions further include, based on the reaction probability:
(i) adding the polymer to a list of potential polymers to be used as at least part of a therapeutic agent,
(ii) removing the polymer from the list of potential polymers to be used as at least part of the therapeutic agent, (iii) ranking the polymer within the list of potential polymers to be used as at least part of the therapeutic agent, or (iv) a combination thereof.

15. A computer-program product tangibly embodied in a non-transitory machine-readable storage medium, including instructions configured to cause one or more data processors to perform actions including:
generating a representation of a polymer having one or more side chains;
performing a molecular-dynamics simulation using the representation, wherein a result of the performance of the molecular-dynamics simulation includes a set of polymer conformations, each polymer conformation of the set of polymer conformations identifying, for each atom in the polymer, a position of the atom;
determining, for each polymer conformation of the set of polymer conformations, one or more spatial characteristics of the polymer while in the polymer conformation, wherein each of the one or more spatial characteristics includes:
a distance between two atoms, each of the two atoms being in a side chain of the one or more polymers or a polymer backbone chain of the polymer;
an angle between three atoms in the polymer; or
a dihedral angle of four atoms in the polymer backbone and the side-chain of the polymer;
identifying, based on the one or more spatial characteristics, an incomplete subset of the set of polymer conformations estimated to undergo one or more reactions of a particular type;
estimating, based on a size of the incomplete subset, a probability of a reaction in which the polymer is a reactant and a particular other molecule is a product; and
outputting the reaction probability.

16. The computer-program product of claim 15, wherein the identification of the incomplete subset includes:
identifying a distance criterion that, when satisfied, indicates that a nitrogen atom within the polymer backbone chain is within a predefined distance from a γ-carbon of the side chain; and
determining that the distance criterion is satisfied for each side-chain conformation in the incomplete subset.

17. The computer-program product of claim 15, wherein the identification of the incomplete subset includes:
identifying an acidity constraint that, when satisfied, indicates that a backbone amide of the polymer backbone chain is acidic, and wherein the acidity constraint is configured to be satisfied when each of at least one backbone dihedral angle of the polymer is within a predefined corresponding range, the one or more spatial characteristics including the at least one backbone dihedral angle; and
determining that the constraint criterion is satisfied for each polymer conformation in the incomplete subset.

18. The computer-program product of claim 15, wherein the identification of the incomplete subset includes:
identifying an accessibility constraint that, when satisfied, indicates that an amide group of the polymer has above-threshold spatial accessibility to bind with a water molecule from a surrounding solvent; and
determining, for each polymer conformation in the incomplete subset, that the accessibility constraint is satisfied based on assessing one or more geometrical characteristics of an intermediate molecule produced when an initial reaction occurs in which the polymer in the polymer conformation is a reactant.

19. The computer-program product of claim 18, wherein determining, for each polymer conformation in the incomplete subset, that the accessibility constraint is satisfied includes:
executing a solvent-inclusive molecular dynamics simulation to simulate the polymer having the polymer conformation in a solvent;
determining, based on one or more results of the execution of the solvent-inclusive molecular dynamics simulation, a water-blocking metric for the polymer conformation; and
determining that the water-blocking metric is within a pre-defined open or closed range of values.

20. The computer-program product of claim 15, wherein the actions further comprise:
determining, based on the reaction probability, to include the polymer in a screen to assess binding affinity for a given target; and
facilitating performance of the screen including the polymer.
